SEQUENCE LISTING (13 sequences)

SEQ ID NO: 1 - protein, 132 aa, human
MLGVLVLGALALAGLGFPDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALVRHIGTDC

SEQ ID NO: 2 - DNA, 396 bp, human
atgcttggggtcctggtccttggcgcgctggccctggccggcctggggttccccgacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgtccgccacattggcaccgactgt

SEQ ID NO: 3 - protein, 132 aa, human
MLGVLVLGALALAGLGFPDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDC

SEQ ID NO: 4 - DNA, 396 bp, human
atgcttggggtcctggtccttggcgcgctggccctggccggcctggggttccccgacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgt

SEQ ID NO: 5 - protein, 480 aa, human
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALVRHIGTDC

SEQ ID NO: 6 - DNA, 1440 bp, human
atgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgtccgccacattggcaccgactgt

SEQ ID NO: 7 - protein, 480 aa, human
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDC

SEQ ID NO: 8 - DNA, 1440 bp, human
atgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgt

SEQ ID NO: 9 - protein, 516 aa, human
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALVRHIGTDCDSGKVDGGDSGSGEPPPSPTPGSTLTPPAVGLVHSG

SEQ ID NO: 10 - DNA, 1548 bp, human
atgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgtccgccacattggcaccgactgtgactccggcaaggtggacggtggcgacagcggctctggcgagcccccgcccagcccgacgcccggctccaccttgactcctccggccgtggggctcgtgcattcgggc

SEQ ID NO: 11 - protein, 516 aa, human
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSG

SEQ ID NO: 12 - DNA, 1548 bp, human
atgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggc

SEQ ID NO: 13 - DNA, 21 bp, Artificial Sequence
  Description of Artificial Sequence Synthetic DNA
aatgtggcgggcaagggccga

(19) United States
(12) Patent Application Publication
(10) Pub. No.: US 2011/0212900 A1
(43) Pub. Date: Sep. 1, 2011

(54) METHOD FOR PROPHYLACTIC AND/OR THERAPEUTIC TREATMENT OF PAIN ASSOCIATED WITH HEMATOPOIETIC CELL TRANSPLANTATION

(75) Inventors: Takayuki IKEZOE, Kochi-shi (JP); Akihito YOKOYAMA, Kochi-shi (JP)

(73) Assignee: National University Corporation Kochi University, Kochi-shi (JP)

(21) Appl. No.: 12/869,477

(22) Filed: Aug. 26, 2010

(30) Foreign Application Priority Data
Feb. 26, 2010 (JP) 2010-042555

(51) Int. Cl. A61K 38/14; A61P 29/00; A61P 37/06; A61P 35/00

(52) U.S. Cl. 514/20.9

(57) ABSTRACT

A method for prophylactic and/or therapeutic treatment of a pain associated with hematopoietic cell transplantation, which comprises the step of administrating thrombomodulin to a mammal.

### TECHNICAL FIELD

The present invention relates to a method for prophylactic and/or therapeutic treatment of a pain associated with hematopoietic cell transplantation. More specifically, the present invention relates to a method for prophylactic and/or therapeutic treatment of a pain associated with hematopoietic cell transplantation comprising the step of administrating thrombomodulin.

### BACKGROUND ART

In blood, three classes of cells exist, i.e., leucocytes, erythrocytes, and platelets. These cells are produced by differentiation and proliferation of hematopoietic stem cells existing in the bone marrow. In hematopoietic malignancies, abnormality is induced in a particular class of cells differentiated from hematopoietic stem cells, resulting in the production of a large amount of hemocytes not having normal functions. As a result, the number of normal blood cells is decreased, and symptoms such as anemia and hemorrhage are developed and infectious diseases are complicated, thereby the disease finally leads to death of the patients. Hematopoietic malignancies mainly consist of leukemia, malignant lymphoma, and multiple myeloma. As for leukemia, 260,000 people suffer from the disease, and 210,000 people the every year in the world. As for malignant lymphoma and multiple myeloma, in total, 410,000 people suffer there from and 240,000 people die every year in the world. In the United States, leukemia, malignant lymphoma and multiple myeloma patients account for 2.9%, 5.3%, and 1% of tumor patients, respectively.

Hematopoietic cell transplantation is a treatment for restoring normal hematopoietic functions by transplanting hematopoietic cells derived from healthy persons to patients with hematopoietic malignancies. In addition to hematopoietic malignancies, hematopoietic cell transplantation has recently been also applied to patients with congenital immunodeficiency or inborn error of metabolism, or to patients with solid tumors as a relief therapy of myelosuppression, the most serious side effect of high-dose chemotherapies and radiotherapies against solid tumors. The hematopoietic cell transplantation is mainly classified into autotransplantation using bone marrow or peripheral stem cells of patients themselves, and allogeneic transplantation using hematopoietic cells from related or unrelated donor. Hematopoietic cells used for the hematopoietic cell transplantation are collected form bone marrow of a donor, peripheral blood of a donor administered with granulocyte colony stimulating factor (G-CSF), or cord blood.

Hematopoietic cell transplantation consists of 1) conditioning regimen, 2) transfusion (transplantation) of hematopoietic cells, and 3) prophylactic and therapeutic treatments of complications. As the conditioning regimen, massive administration of chemotherapeutic agents) such as cyclophosphamide and total body irradiation (TBI) are performed 7 to 10 days before the transplantation causing bone marrow cells death in a patient who is to receive the hematopoietic cell transplantation. Then, hematopoietic cells obtained from a donor are transfused. As for the number of cells required for the transplantation, about 3×10⁸ of nucleated cells per 1 kg of body weight are preferably transplanted in the case of bone marrow transplantation, 2×10⁶ or more of CD34-positive cells per 1 kg of body weight are preferably transplanted in the case of peripheral stem cell transplantation, and 2×10⁷ or more of cells per 1 kg of body weight are preferably transplanted in the case of cord blood transplantation.

Post-transplantation complications are often developed in three months after the transplantation of hematopoietic cells, and for successful transplantation therapy, it is important to overcome the complications which are intensively developed in one month after the transplantation. In addition to infections, hemorrhage, and anemia, that are caused by hematopenia, graft versus host disease (GVHD), sinusoidal obstruction syndrome (SOS), and transplantation-associated thrombotic microangiopathy (TAM) may be developed as the conplications associated with transplantation.

GVHD is mainly classified into an acute type normally developed within 100 days after the transplantation, and a chronic type of which development time is not set criteria. The acute GVHD is defined in that dysfunction is observed in at least one organ among the skin, liver, and gut, whilst possibility of GVHD-like diseases is denied, and shows symptoms including maculopapular rash, jaundice, diarrhea, and severe abdominal pain. In the chronic GVHD, inflammations accompanying pain are developed in the oral cavity, joints, and eyeballs in addition to the symptoms with the acute type. For prophylactic treatment of GVHD, sole use of cyclosporin or methotrexate, combined use of cyclosporin and methotrexate, combined use of tacrolimus and methotrexate, and combined use of cyclosporin and mycophenolic acid have been mainly applied. Combined use of a steroid together with the above medicaments has also been employed. For therapeutic treatment of GVHD, administration of a steroid such as methylprednisolone, or use of mycophenolic acid or antithymocyte globulin has been employed (Non-patent document 1).

In SOS, symptoms such as weight gain from an unknown reason (>2%), ascites, right hypochondrium pain, hepatomegaly, and jaundice (or total bilirubin (TB) ≧2 mg/dL) are developed by ischemia and necrosis of hepatocytes due to obstruction of sinusoid generally one to two weeks after the transplantation. Clinically, the syndrome is diagnosed based on any of the Seattle criteria, modified Seattle criteria, and Baltimore criteria. In the Seattle criteria, it is defined that at least two symptoms are observed within 30 days after the transplantation among 1) jaundice, 2) hepatomegaly with pain in right upper quadrant of abdomen, and 3) ascites and/or weight gain from an unknown reason. In the modified Seattle criteria, it is defined that at least two symptoms are observed within 20 days after the transplantation among 1) jaundice (or TB a ≧2 mg/dL), 2) hepatomegaly with pain in right upper quadrant of abdomen, and 3) 2% or more of weight gain due to water retention. In the Baltimore criteria, it is defined that, in addition to jaundice (or TB a ≧2 mg/dL) developed within 21 days after the transplantation, at least two symptoms are observed among 1) hepatomegaly (with pain), 2) 5% or more of weight gain, and 3) ascites (Non-patent document 2).

As risk factors for development of SOS after the transplantation, hepatopathy due to chemotherapy before the transplantation, infectious diseases, or virus hepatitis, hepatopathy due to radiotherapy or chemotherapeutic agent (for example, busulfan) used in the conditioning regimen of hematopoietic cell transplantation, immunological mechanism of allogeneic transplantation, and complications of infectious diseases due to use of an immunosuppressant and the like are known. Since increase of inflammatory cytokines such as TNF-α, IL-6 and IL-8 as well as clotting abnormality are developed in patients having SOS, the syndrome has been considered to be a thrombotic disease developed on the basis of vascular endothelial cell disorders. SOS formally known as hepatic veno-occlusive disease (or veno-occlusive disease, VOD), however, since this pathologic syndrome relates to obstruction of sinusoid, and obstruction of the hepatic vein is not essential, the syndrome has recently been preferred the name of SOS which more directly reflects the pathologic conditions.

Although administrations of heparin, prostaglandin E1, ursodeoxycholic acid, pentoxifylline, and antithrombin III (ATIII) and the like have been used for prophylaxis of SOS, it is almost far from satisfactory results are obtained at present by the therapies. As therapeutic treatments of SOS, symptomatic therapies are applied such as administration of a diuretic, ATIII, or fresh frozen plasma (FFP), as well as a fibrinolytic therapy with tissue plasminogen activator (tPA). However, since the tPA therapy is used in an early stage after the transplantation where the number of platelet is reduced, critical side reactions such as intracranial hemorrhage have also been often reported, and moreover, despite such dangerous side reactions, an effective rate is as low as about 30% at present. Severity of SOS is classified in mild, moderate, and severe conditions. Most of mild SOS is naturally cured, whilst severe SOS leads to a mortality of 90% or higher.

TAM is a thrombotic microangiopathy (TMA) associated with transplantation, and is a syndrome characterized by hemolytic anemia, consumption thrombocytopenia, and organ dysfunction due to microcirculation obstruction. Pathologically, platelet thrombus formation is observed in microvessels. TMA is proposed to be diagnosed with the criteria of BMT-CTN (Blood and Marrow Transplants Clinical Trials Network) in North America or EBMT-ELN (European Group for Blood and Marrow Transplantation-European Leukemia Network) in Europe (Non-patent document 3).

According to the criteria of BMT-CTN in North America, criteria of 1) presence of schistocytes (two or more in one high power field), 2) increase of LDH (above institutional baseline), 3) presence of renal or neurogic dysfunction without other explanations, and 4) negative direct or indirect Coombs tests are used. According to the criteria of EBMT-ELT in Europe, diagnosis is carried out by using criteria of 1) increase in schistocytes in peripheral blood (more than 4%), 2) prolonged or progressive thrombocytopenia in a newly developing patient (lower than 50,000/μL, or decrease of 50% or more compared with that observed before the onset), 3) quickly appeared and prolonged increase in LDH, 4) decrease in hemoglobin or increase in erythrocyte transfusion, and 5) decrease in serum haptoglobin.

As the clinical symptoms, TMA exhibits microangiopathic hemolytic anemia (MAHA), central nerve obstruction, renal disorder, ischemic enterocolitis, and the like. Diarrhea, pyrexia, jaundice, central nerve symptoms, diarrhea, and the like are the major symptoms, and distinction from acute GVHD may sometimes raise a problem. Increase in schistocytes, duration of high LDH level, and progressive thrombocytopenia which does not respond to platelet transfusion are observed, but abnormality in characteristic laboratory findings indicating vascular endothelial cell dysfunctions is often unclear. Since cyclosporin and tacrolimus used for prophylactic and therapeutic treatments of GVHD are aggravating factors for TMA, reducing the dose or discontinuing the medication is considered, however, such means are not always effective. Further, although effectiveness of defibrotide is reported, any prophylactic or therapeutic treatment has not yet been established (Non-patent document 1).

In patients who received hematopoietic cell transplantation, pain is often complicated, and low QOL of the patients poses a problem. In GVHD, abdominal pain, pain of conjunctiva and eyeball, tenderness of skeletal muscles, pain in the oral cavity, and arthralgia are observed. In TMA, painful rash and diarrhea accompanied by abdominal pain are observed. In VOD, hypochondriac pain accompanied by hepatomegaly is observed. Conditioning regimen of the transplantation and use of an immunosuppressant or the like after the transplantation also develop pain. For example, a chemotherapeutic drug or radiotherapy used as a conditioning regimen of hematopoietic cell transplantation provides erythema accompanied by pain in the palm, end phalanx, sole, scrotum, and the like in an early stage of the transplantation. Use of a calcineurin inhibitor such as tacrolimus is reported to develop intolerable superficial and intractable pain in lower extremities and the like, i.e., calcineurin inhibitor-induced pain syndrome (CIPS) (Non-patent document 4). Further, antithymocyte globulin and alemtuzumab, which have prophylactic and therapeutic effects against GVHD, induces arthralgia and headache. As described above, hematopoietic cell transplantation-associated pain is also developed by hematopoietic cell transplantation therapy and prophylactic and therapeutic treatments of complications of hematopoietic cell transplantation. QOL of patients developing these pains is not sufficiently high, and therefore, it is desired to further improve transplantation results by effectively preventing or treating the complications through improvements in hematopoietic cell transplantation methods.

Thrombomodulin has been known as a substance that acts to specifically bind to thrombin so as to inhibit the blood coagulation activity of thrombin, and at the same time, exerts anticoagulant activity so as to significantly promote the ability of thrombin to activate the protein C. It has also been known that thrombomodulin exerts to prolong the clotting time by thrombin, or suppresses platelet aggregation by thrombin. The protein C is a vitamin K-dependent protein that plays an important role in a blood coagulation and fibrinolysis, and activated by the action of thrombin to be converted as activated protein C. It has been known that the activated protein C inactivates activated blood coagulation factor V and activated blood coagulation factor VIII in vivo, and is involved in generation of a plasminogen activator having thrombolytic action (Non-patent document 5). Accordingly, it has been considered that thrombomodulin promotes the activation of the protein C by thrombin, and therefore is useful as an anticoagulant or a thrombolytic agent. It has also been reported that, in an animal experiment, thrombomodulin is effective for therapy or prophylaxis of diseases associated with hypercoagulable state (Non-patent document 6).

Thrombomodulin was first discovered and obtained as a glycoprotein expressed on the vascular endothelial cells of various animal species including humans, and then successfully cloned. Specifically, a gene of a human thrombomodulin precursor including a signal peptide was cloned from a human lung cDNA library by genetic engineering techniques and the entire gene sequence of thrombomodulin was analyzed, and as a result, an amino acid sequence consisting of 575 residues containing a signal peptide (in general, 18 amino acid residues are exemplified) was revealed (Patent document 1). It is known that a mature thrombomodulin, from which the signal peptide is cleaved, is composed of 5 regions, namely, an N-terminal region (amino acid residues 1 to 226, these positions are defined under an assumption that the signal peptide consists of 18 amino acid residues, and the same shall apply to the following descriptions), a region having six EGF-like structures (amino acid residues 227 to 462), an O-linked glycosylation region (amino acid residues 463 to 498), a transmembrane region (amino acid residues 499 to 521), and an cytoplasmic region (amino acid residues 522 to 557), from the N-terminal side of the mature peptide. It is also known that a partial protein having the same activity as that of the entire length thrombomodulin (i.e., a minimal active unit) is mainly consisting of the 4th, 5th, and 6th EGF-like structures from the N-terminal side in the region having six EGF-like structures (Non-patent document 2).

The entire length thrombomodulin is hardly dissolved in the absence of a surfactant, and addition of a surfactant is essential for manufacturing an entire thrombomodulin preparation. A soluble thrombomodulin is also available that can be fully dissolved even in the absence of a surfactant. The soluble thrombomodulin may be prepared by removing at least a part of the transmembrane region or the entire transmembrane region. For example, it has been confirmed that a soluble thrombomodulin consisting of only 3 regions, namely, the N-terminal region, the region having six EGF-like structures, and the O-linked glycosylation region (i.e., a soluble thrombomodulin having an amino acid sequence consisting of amino acid residues 19 to 516 of SEQ ID NO: 9) can be obtained by applying recombination techniques, and that the resulting recombinant soluble thrombomodulin has the same activity as that of an entire thrombomodulin (Patent document 1). Some other reports are also available regarding soluble thrombomodulins (Patent documents 2 to 9). A human urine-derived soluble thrombomodulin and the like are also exemplified as native thrombomodulins (Patent documents 10 and 11).

As recognized in many cases, as a result of spontaneous mutations or mutations occurring at the time of obtainment, polymorphic mutations have been found in the human genes. At present, thrombomodulin proteins in which the amino acid at the position 473 of human thrombomodulin precursor having the amino acid sequence consisting of 575 amino acid residues is converted to Val or Ala have been identified. In the nucleotide sequence encoding the amino acid sequence, this variation of amino acid residue corresponds to mutation to T or C at the position 1418 (Non-patent document 7). However, the two types of thrombomodulins are completely identical in terms of their activity and physicochemical properties, and it can be considered that they are substantially identical.

As for use of thrombomodulin, there is conventionally expected use for therapeutic and prophylactic treatments of diseases such as, for example, myocardial infarction, thrombosis (for example, cerebral thrombosis of an acute stage or chronic stage, acute or chronic peripheral thrombosis of an artery or vein, and the like), embolism (for example, cerebral embolism of an acute stage or chronic stage, acute or chronic peripheral embolism of an artery or vein and the like), peripheral vascular occlusive diseases (for example, Buerger's disease, Raynaud's disease, and the like), arteriosclerosis obliterans, dysfunction secondary to heart surgery, complications of organ transplantation, disseminated intravascular coagulation (DIC), angina pectoris, transient ischemic attack, gestosis, deep venous thrombosis (DVT), and the like. As applications to diseases other than those accompanied by hypercoagulable states such as thrombosis and DIC, applicable disease include hepatopathy (Patent document 12), bone resorption (Patent document 13), wound healing (Patent document 14), and the like. Furthermore, as uses of thrombomodulin as a combination with other active ingredients, wound healing (Patent document 15), protection of brain tissues (Patent document 16) and the like are disclosed.

VOD is described as a target use of thrombomodulin for hematopoietic cell transplantation-associated complications (Patent document 17). This patent document described that elevations of GPT and bilirubin were suppressed by administering 1 mg/kg or 3 mg/kg of thrombomodulin in a drug-induced VOD model induced by administering monocrotaline to a beagle by referring to Non-patent document 9.

Further, TAM is described as a target use of thrombomodulin for hematopoietic cell transplantation-associated complications (Patent document 18). This patent document described that the effect of thrombomodulin was investigated using a hemolysis model induced by administrating Ricinus communis agglutinin I, RCA120 and a lipopolysaccharide (LPS).

### PRIOR ART REFERENCES

### Patent Documents

Patent document 1: Japanese Patent Unexamined Publication (Kokai) No. 64-6219
Patent document 2: Japanese Patent Unexamined Publication No. 5-213998
Patent document 3: Japanese Patent Unexamined Publication No. 2-255699
Patent document 4: Japanese Patent Unexamined Publication No. 3-133380
Patent document 5: Japanese Patent Unexamined Publication No. 3-259084
Patent document 6: Japanese Patent Unexamined Publication No. 4-210700
Patent document 7: WO92/00325
Patent document 8: WO92/03149
Patent document 9: WO93/15755
Patent document 10: Japanese Patent Unexamined Publication No. 3-86900
Patent document 11: Japanese Patent Unexamined Publication No. 3-218399
Patent document 12: Japanese Patent Unexamined Publication No. 8-3065
Patent document 13: Japanese Patent Unexamined Publication No. 8-301783
Patent document 14: Japanese Patent Unexamined Publication No. 9-20677
Patent document 15: U.S. Pat. No. 5,976,523
Patent document 16: U.S. Pat. No. 5,827,832
Patent document 17: U.S. Pat. No. 5,916,874
Patent document 18: WO2002/028416

### Non-Patent Documents

Non-patent document 1: Hematopoietic cell transplantation guideline GVHD, Japan Society for Hematopoietic Cell Transplantation, 2008
Non-patent document 2: Sakurai C., Complications in digestive organ and liver, In: Kanda Y., Fundamentals and clinics of hematopoietic stem cell transplantation (2nd volume), 1st edition, 77-82, 2008, Iyaku (Medicine and Drug) Journal Co., Ltd., Tokyo
Non-patent document 3: Matsumoto M., Journal of Thrombosis and Haemostasis, 19(3), 363-5, 2008
Non-patent document 4: Fujii N. et al, Int. J. Hematol., 83, 459-61, 2006
Non-patent document 5: Suzuki K., Progress of Medicine, 125:901, 1983
Non-patent document 6: Gomi K. et al., Blood, 75, 1396-1399, 1990
Non-patent document 7: Zushi M. et al., J. Biol. Chem., 246, 10351-10353, 1989
Non-patent document 8: Wen D. Z. et al., Biochemistry, 26, 4350-4357, 1987
Non-patent document 9: Epstein R. B. et al., Transplant, 54(1), 12-16, 1992

### SUMMARY OF THE INVENTION

### Object to be Achieved by the Invention

An object of the present invention is to provide a medicament or method for effective prophylactic or therapeutic treatment of a pain associated with hematopoietic cell transplantation.

Another object of the present invention is to provide a highly therapeutically effective medicament for improving hematopoietic ability for use at the time of hematopoietic cell transplantation, and a method for normalizing peripheral blood count for use at the time of hematopoietic cell transplantation in a hematopoietic malignancy patient.

Still another object of the present invention is to provide a highly therapeutically effective medicament or method for therapeutic treatment and/or improvement of pathological conditions of a hematopoietic malignancy patient who is to undergo hematopoietic cell transplantation or a hematopoietic malignancy patient who underwent hematopoietic cell transplantation.

### Means for Achieving the Objects

Treatment results of hematopoietic cell transplantation have been improving in recent years with combinations of novel chemotherapeutic agents or medicaments and the like. However, mortality of patients developing complications is still high. Further, strong pain is developed in the eyeball, oral cavity, abdomen, and the like, and accordingly QOL of patients is not considered to be sufficiently high. Therefore, it is desired to further improve transplantation results by effectively prevent or treat complications with improvement of hematopoietic cell transplantation methods.

The inventors of the present invention focused on prophylactic and/or therapeutic treatment of a pain associated with hematopoietic cell transplantation, and conducted various researches to find a satisfactory medicament. As a result, they unexpectedly found that administration of thrombomodulin achieved remarkable improvements of clotting abnormality, edema and weight gain associated with hematopoietic cell transplantation, and moreover achieved improvement of pain. They also found that the ameliorating effect of pain was a fast-acting effect compared with that of clotting abnormality, edema, and weight gain, and the effect successfully contributed to improvement in QOL for patients undergoing hematopoietic cell transplantation patients. They further found that hematopoietic activity was successfully restored by administering thrombomodulin. The inventors accomplished the present invention on the basis of each of these findings.

The present invention thus relates to a medicament for prophylactic and/or therapeutic treatment of a pain associated with hematopoietic cell transplantation, which comprises thrombomodulin as an active ingredient. By administering the medicament of the present invention undergoing hematopoietic cell transplantation, which is expected as a curative treatment of hematopoietic malignancies, it becomes possible to improve hematopoietic activity of a patient who undergo hematopoietic cell transplantation. It also becomes possible to normalize myelogram and peripheral blood count of a hematological malignancy patient who underwent hematopoietic cell transplantation, and thereby more effective therapeutic treatment and/or improvement of the hematological malignancy is achievable.

Specifically, the present invention includes the followings:
[1] A medicament for prophylactic and/or therapeutic treatment of a pain associated with hematopoietic cell transplantation, which comprises thrombomodulin as an active ingredient;
[1-2] The medicament for augmenting prophylactic and/or therapeutic effect on a pain associated with hematopoietic cell transplantation, which comprises thrombomodulin as an active ingredient;
[2] The medicament according to [1] or [1-2] mentioned above, wherein the thrombomodulin is a soluble thrombomodulin;
[3] The medicament according to any one of [1] to [2] mentioned above, wherein the thrombomodulin is a peptide obtainable from a transformed cell prepared by transfecting a host cell with a DNA coding for the amino acid sequence of SEQ ID NO: 9 or SEQ ID NO: 11;
(when item numbers are indicated with a range such as “[1] to [2] mentioned above”, and when an item having a subnumber such as [1-2] is placed in said range, it is meant that the item assigned with the subnumber is also cited, and the same shall apply to the following descriptions)
[3-2] The medicament according to any one of [1] to [2] mentioned above, wherein the thrombomodulin is a peptide obtainable from a transformed cell prepared by transfecting a host cell with a DNA coding for the amino acid sequence of SEQ ID NO: 1, 3, 5, 7, 9, or 11;
[3-3] The medicament according to any one of [1] to [2] mentioned above, wherein the thrombomodulin is a peptide obtainable from a transformed cell prepared by transfecting a host cell with a DNA coding for the amino acid sequence of SEQ ID NO: 1 or SEQ ID NO: 3;
[3-4] The medicament according to any one of [1] to [2] mentioned above, wherein the thrombomodulin is a peptide obtainable from a transformed cell prepared by transfecting a host cell with a DNA coding for the amino acid sequence of SEQ ID NO: 5 or SEQ ID NO: 7;
[4] The medicament according to any one of [1] to [3] mentioned above, wherein the thrombomodulin is a peptide having the sequence from the position 19 to 516 in either of SEQ ID NO: 9 or SEQ ID NO: 11, or a peptide having an amino acid sequence comprising substitution, deletion, or addition of one or more amino acid residues of the amino acid sequence of the aforementioned peptide and having thrombomodulin activity;
[4-2] The medicament according to any one of [1] to [2] and [3-3] mentioned above, wherein the thrombomodulin is a peptide having the sequence from the position 19 to 132 in either of SEQ ID NO: 1 or SEQ ID NO: 3, or a peptide having an amino acid sequence comprising substitution, deletion, or addition of one or more amino acid residues of the amino acid sequence of the aforementioned peptide and having thrombomodulin activity;
[4-3] The medicament according to any one of [1] to [2] and [3-4] mentioned above, wherein the thrombomodulin is a peptide having the sequence from the position 19 to 480 in either of SEQ ID NO: 5 or SEQ ID NO: 7, or a peptide having an amino acid sequence comprising substitution, deletion, or addition of one or more amino acid residues of the amino acid sequence of the aforementioned peptide and having thrombomodulin activity;
[4-4] The medicament according to any one of [1] to [2] and [3] mentioned above, wherein the thrombomodulin is a peptide having the sequence from the position 19 to 516 either of SEQ ID NO: 9 or SEQ ID NO: 11;
[4-5] The medicament according to any one of [1] to [2] and [3] mentioned above, wherein the thrombomodulin is a peptide having the sequence from the position 19 to 516, 19 to 515, 17 to 516, or 17 to 515 in either of SEQ ID NO: 9 or SEQ ID NO: 11, or a peptide having an amino acid sequence comprising substitution, deletion, or addition of one or more amino acid residues of the amino acid sequence of the aforementioned peptide and having thrombomodulin activity;
[4-6] The medicament according to any one of [1] to [2] and [3] mentioned above, wherein the thrombomodulin is a peptide having the sequence from the position 19 to 516, 19 to 515, 17 to 516, or 17 to 515 in either of SEQ ID NO: 9 or SEQ ID NO: 11, or a mixture thereof;
[4-7] The medicament according to any one of [1] to [2] and [3] mentioned above, wherein the thrombomodulin is a peptide having the sequence from the position 19 to 516, 19 to 515, 17 to 516, or 17 to 515 in either of SEQ ID NO: 9 or SEQ ID NO: 11, or a mixture thereof;
[5] The medicament according to any one of [1] to [4-7] mentioned above, wherein the hematopoietic cell transplantation is hematopoietic cell transplantation for a hematological malignancy;
[6] The medicament according to any one of [1] to [5] mentioned above, wherein the pain associated with hematopoietic cell transplantation is a pain developed with any one disease selected from the group consisting of graft versus host disease, sinusoidal obstruction syndrome, and transplantation-associated thrombotic microangiopathy;
[6-2] The medicament according to any one of [1] to [5] mentioned above, wherein the pain associated with hematopoietic cell transplantation is a pain developed with graft versus host disease;
[6-3] The medicament according to any one of [1] to [5] mentioned above, wherein the pain associated with hematopoietic cell transplantation is a pain developed with sinusoidal obstruction syndrome;
[6-4] The medicament according to any one of [1] to [5] mentioned above, wherein the pain associated with hematopoietic cell transplantation is a pain developed with transplantation-associated thrombotic microangiopathy;
[7] The medicament according to any one of [1] to [6-4] mentioned above, wherein the pain associated with hematopoietic cell transplantation is developed in any one region selected from the group consisting of the eyeball, oral cavity, abdomen and hypochondrium;
[7-2] The medicament according to any one of [1] to [6-4] mentioned above, wherein the pain associated with hematopoietic cell transplantation is developed in the eyeball;
[7-3] The medicament according to any one of [1] to [6-4] mentioned above, wherein the pain associated with hematopoietic cell transplantation is developed in the oral cavity;
[7-4] The medicament according to any one of [1] to [6-4] mentioned above, wherein the pain associated with hematopoietic cell transplantation is developed in the abdomen.
[7-5] The medicament according to any one of [1] to [6-4] mentioned above, wherein the pain associated with hematopoietic cell transplantation is developed in the hypochondrium.
[8] A medicament for prophylactic and/or therapeutic treatment of a pain associated with hematopoietic cell transplantation, which comprises the medicament according to any one of [1] to [7-5] mentioned above and at least one therapeutic agent selected from the group consisting of an immunosuppressant and a therapeutic agent for hematological malignancy in combination;
[8-2] A medicament for prophylactic and/or therapeutic treatment of a pain associated with hematopoietic cell transplantation, which comprises the medicament according to any one of [1] to [7-5] mentioned above and an immunosuppressant in combination;
[8-3] A medicament for prophylactic and/or therapeutic treatment of a pain associated with hematopoietic cell transplantation, which comprises the medicament according to any one of [1] to [7-5] mentioned above, an immunosuppressant and a therapeutic agent for hematological malignancy in combination;
[8-4] The medicament for boosting prophylactic and/or therapeutic effect on a pain associated with hematopoietic cell transplantation, which comprises the medicament according to any one of [1] to [7-5] mentioned above and at least one therapeutic agent selected from the group consisting of an immunosuppressant and a therapeutic agent for hematological malignancy in combination;
[8-5] The medicament for boosting prophylactic and/or therapeutic effect on a pain associated with hematopoietic cell transplantation, which comprises the medicament according to any one of [1] to [7-5] mentioned above and an immunosuppressant in combination;
[8-6] A medicament for boosting prophylactic and/or therapeutic effect on a pain associated with hematopoietic cell transplantation, which comprises the medicament according to any one of [1] to [7-5] mentioned above, an immunosuppressant and a therapeutic agent for hematological malignancy in combination;
[8-7] A medicament for prophylactic and/or therapeutic treatment of a pain associated with hematopoietic cell transplantation, which comprises the medicament according to any one of [1] to [7-5] mentioned above and at least one therapeutic agent selected from the group consisting of an immunosuppressant, a therapeutic agent for hematological malignancy, and a prophylactic and/or therapeutic agent for a complication associated with hematopoietic cell transplantation in combination;
[8-8] A medicament for boosting prophylactic and/or therapeutic effect on a pain associated with hematopoietic cell transplantation, which comprises the medicament according to any one of [1] to [7-5] mentioned above and at least one therapeutic agent selected from the group consisting of an immunosuppressant, a therapeutic agent for hematological malignancy, and a prophylactic and/or therapeutic agent for a complication associated with hematopoietic cell transplantation in combination;
[9] The medicament according to any one of [1] to [7-5] mentioned above, which is for administration together with at least one therapeutic agent selected from the group consisting of an immunosuppressant and a therapeutic agent for hematological malignancy;
[9-2] The medicament according to any one of [1] to [7-5] mentioned above, which is for administration together with an immunosuppressant;
[9-3] The medicament according to any one of [1] to [7-5] mentioned above, which is for administration together with an immunosuppressant and a therapeutic agent for hematological malignancy;
[9-4] The medicament according to any one of [1] to [7-5] mentioned above, which is for administration together with at least one therapeutic agent selected from the group consisting of an immunosuppressant, a therapeutic agent for hematological malignancy, and a prophylactic and/or therapeutic agent for a complication associated with hematopoietic cell transplantation;
[9-5] The medicament according to any one of [1] to [7-5] mentioned above, which is for administration together with an immunosuppressant, a therapeutic agent for hematological malignancy, and a prophylactic and/or therapeutic agent for a complication associated with hematopoietic cell transplantation;
[10] A medicament for improving hematopoietic ability for use at the time of hematopoietic cell transplantation for hematological malignancy, which comprises thrombomodulin as an active ingredient;
[11] Use of thrombomodulin for manufacture of a medicament for prophylactic and/or therapeutic treatment of a pain associated with hematopoietic cell transplantation;
[11-2] The use of thrombomodulin for manufacture of a medicament for prophylactic and/or therapeutic treatment of a pain associated with hematopoietic cell transplantation, which is characterized by the characteristic of any one of [1] to [11] mentioned above;
[12] A method for prophylactic and/or therapeutic treatment of a pain associated with hematopoietic cell transplantation, which comprises the step of administrating thrombomodulin to a mammal;
[12-2] The method for prophylactic and/or therapeutic treatment of a pain associated with hematopoietic cell transplantation, which comprises the step of administrating thrombomodulin to a mammal, and is characterized by the characteristic of any one of [1] to [11] mentioned above;
[13] A method for prophylactic and/or therapeutic treatment of a pain associated with hematopoietic cell transplantation, which comprises the step of administrating thrombomodulin to a patient having a normal bilirubin value.
[13-2] The method according to [13] mentioned above, wherein the pain is a hypochondrial pain;
[14] A method for prophylactic and/or therapeutic treatment of a pain associated with hematopoietic cell transplantation, which comprises the step of administrating thrombomodulin to a patient for whom defibrotide has no ameliorating effect of pain;
[14-2] The method according to [14] mentioned above, wherein the pain is a hypochondrial pain;
[15] A method for prophylactic and/or therapeutic treatment of a pain associated with hematopoietic cell transplantation, which comprises the step of administrating thrombomodulin to a patient having a normal bilirubin value for whom defibrotide shows no ameliorating effect of pain;
[15-2] The method according to [15] mentioned above, wherein the pain is a hypochondrial pain.

### Effect of the Invention

With the medicament of the present invention comprising thrombomodulin, a pain associated with hematopoietic cell transplantation can be prevented and/or treated. According to the method of the present invention, myelogram after hematopoietic cell transplantation and/or peripheral blood count after hematopoietic cell transplantation can be effectively normalized, and it becomes possible to more effectively treat and/or improve hematological malignancy.

### BRIEF DESCRIPTION OF THE DRAWINGS

FIG. 1 shows the time course of various laboratory test values over time in a Ph-positive ALL patient after hematopoietic cell transplantation. The results are indicated with a definition that the day of the hematopoietic cell transplantation is Day 0. Administration periods of methylprednisolone (mPSL), defibrotide (25 mg/kg), and TMD123 (rTM, 380 units/kg [0.06 mg/kg]) are indicated with thick block frame border. It is indicated that administration of mPSL was started at a dose of 1 mg/kg, and the dose was decreased stepwise to 0.5 mg/kg and 0.25 mg/kg. The clays of ATIII administration (1,500 units/day), granulocyte colony stimulating factor (G-CSF) administration, and platelet transfusion (PL) are indicated with arrows. It is shown that, on the second days of the rTM administration, weight gain indicating advance of edema was observed, but the body weight began to decrease on the third day of the rTM administration and decreased thereafter.

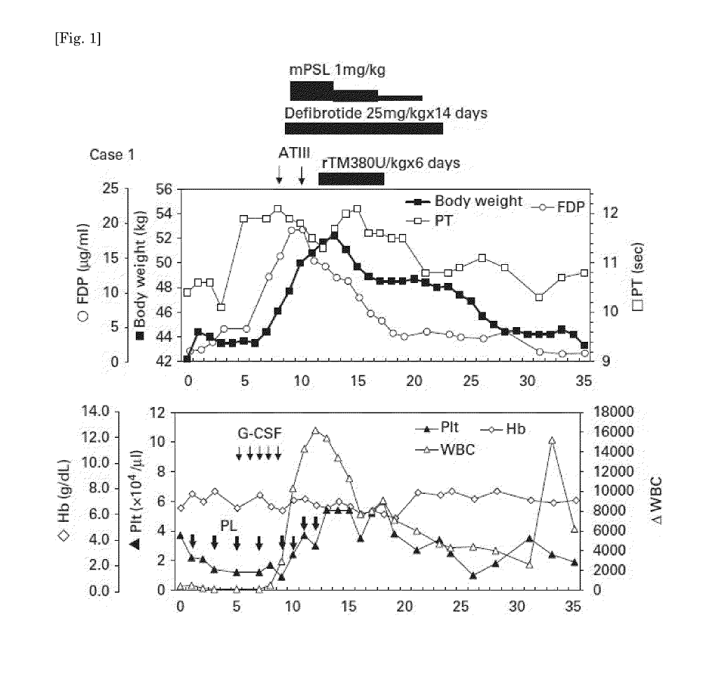

▪ Body weight (kg) represents body weight. ◯ FDP (μg/ml) represents fibrinogen and fibrin degradation product). □ PT (sec) represents prothrombin time. ▴ Plt (×10⁴/μl) represents platelet count. ⋄ Hb (g/dL) represents hemoglobin. Δ WBC represents white blood cell count (/μl).

FIG. 2 shows the time course of various laboratory test values over time in an ATL patient after hematopoietic cell transplantation. The results are indicated with a definition that the day of the hematopoietic cell transplantation is Day 0. Administration period of TMD123 (rTM, 380 units/kg [0.06 mg/kg]) is indicated with thick black frame border. The days of ATIII administration (1,500 units/day), G-CSF administration, and platelet transfusion (PL) are indicated with arrows. It is shown that weight gain was disappeared by the rTM administration, and the body weight began to decrease on the sixth day of the rTM administration and decreased thereafter. Body weight, FDP, PT, Plt, Hb, and WBC have the same meanings as those of FIG. 1, respectively.

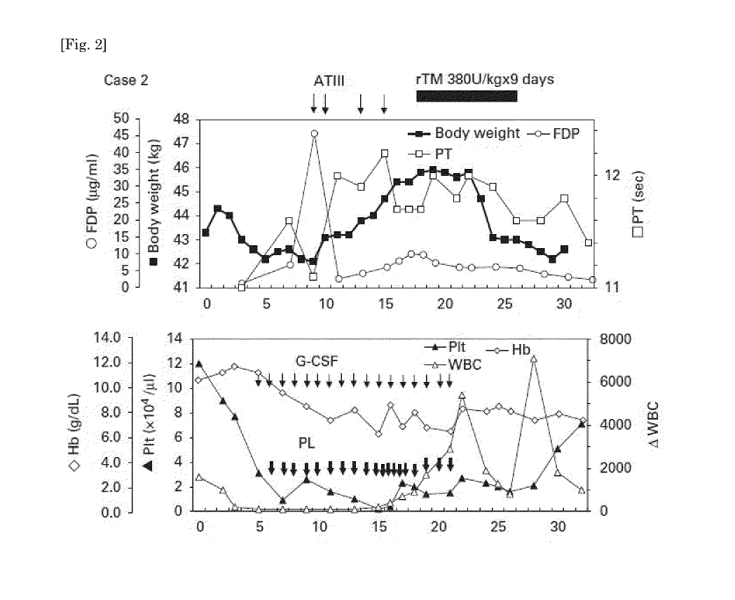

### MODES FOR CARRYING OUT THE INVENTION

Hereafter, the present invention will be specifically explained.

In the present invention, specific examples of the hematopoietic cell transplantation include, as mentioned above, hematopoietic cell transplantation as a therapeutic treatment of hematological malignancy as well as hematopoietic cell transplantation as a relief therapy of congenital immunodeficiency, inborn error of metabolism, and myelosuppression, which is the greatest adverse drug reaction of high-dose chemotherapy and radiotherapy for solid tumors. The medicament of the present invention can also be used for the aforementioned types of hematopoietic cell transplantation.

In the present invention, preferred examples of the method of hematopoietic cell transplantation include 1) transfusion of bone marrow cells harvested from a donor to a patient, 2) transfusion of only CD34-positive cells isolated from peripheral blood harvested from a donor administered with G-CSF, and 3) transfusion of once frozen and thawed cord blood.

In the present invention, preferred examples of the donor include a hematological malignancy patient oneself, a human leukocyte antigen (HLA)-matched or substantially matched close relative of a hematological malignancy patient, and an HLA-matched or substantially matched non-relative.

In the present invention, examples of the peripheral blood count include red blood cell count, white blood cell count, and platelet count in the whole blood. The normalization of peripheral blood count is not particularly limited, so long as the peripheral blood count is increased by the normalization so that it can be judged that the pathological conditions of hematological malignancy are improved.

The normalized peripheral blood count is indicated with, for example, red blood cell count, white blood cell count, and platelet count in the whole blood after the transplantation. As for the lower limit of the peripheral red blood cell count, the count is preferably 3,600,000/μL or more, more preferably 3,800,000/μL or more, still more preferably 4,000,000/μL, in the case of male. As for the upper limit of the peripheral red blood cell count, the count is preferably 5,800,000/μL or less, more preferably 5,400,000/μL or less. In the case of female, as for the lower limit of the peripheral red blood cell count, the count is preferably 3,300,000/μL or more, more preferably 3,600,000/μL or more. As for the upper limit, the peripheral red blood cell count is preferably 5,200,000/μL or less, more preferably 4,900,000/μL or less.

As for the lower limit of the peripheral white blood cell count, the count is preferably 4,000/μL or more. As for the upper limit of the peripheral white blood cell count, the count is preferably 14,000/μL or less. As for the lower limit of the peripheral platelet count, the count is preferably 80,000/μL or more, more preferably 120,000/μL or more, most preferably 150,000/μL. As for the upper limit of the peripheral platelet count, the count is preferably 500,000/μL or less, more preferably 350,000/μL or less. As for time for recovering the peripheral blood count to a level within the aforementioned ranges, it should be preferably recovered by 6 months, more preferably 3 months, still more preferably 1 month, after the transplantation. A preferred embodiment of the normalization of the peripheral blood count includes maintaining the peripheral blood count within the aforementioned ranges.

In the present invention, examples of augumenting therapeutic and/or improving effect of hematopoietic cell transplantation on a hematological malignancy include enhancing the therapeutic and/or improving effect on the hematological malignancy or increasing probability of cure and/or improvement of the hematological malignancy by using the medicament of the present invention at the time of treating the hematological malignancy with hematopoietic cell transplantation. Examples also include treating and/or improving the hematological malignancy, per se.

The medicament of the present invention can be administered to a patient who is to undergo hematopoietic cell transplantation or to a patient who has undergone hematopoietic cell transplantation. As for the time of the administration of the medicament of the present invention, the medicament may be administered before a patient receives the hematopoietic cell transplantation or after a patient receives the hematopoietic cell transplantation. When the medicament is administered to a patient who is to undergo hematopoietic cell transplantation, the medicament may be administered by the time of the hematopoietic cell transplantation, and the medicament is preferably administered by the time of the start of a conditioning regimen of the hematopoietic cell transplantation. When the medicament is administered to a patient who has already undergone hematopoietic cell transplantation, the medicament may be administered after the patient undergoes the hematopoietic cell transplantation, and the medicament is preferably administered by the time of a point when diagnosis criteria of each complication are met. Furthermore, the medicament of the present invention may be administered to a patient under hematopoietic cell transplantation. In this case, the medicament may be administered at the same time as carrying out the hematopoietic cell transplantation.

The patient to be administered with the medicament of the present invention is not particularly limited, so long as the patient is to undergo hematopoietic cell transplantation or a patient who has undergone hematopoietic cell transplantation, or a patient under hematopoietic cell transplantation. The patient is preferably a hematological malignancy patient.

The hematopoietic cell transplantation is preferably hematopoietic cell transplantation performed for a hematological malignancy, i.e., transplantation of hematopoietic cells in a patient with the hematological malignancy.

In the present invention, although the hematological malignancy patient is not particularly limited, so long as the patient is a hematological malignancy patient whose hematopoietic activity is highly obstructed, specific examples of the patient include patients of leukemia, malignant lymphoma, multiple myeloma, and related diseases thereof, and a preferred example is an acute leukemia patient. A chronic leukemia patient may also be preferred. Examples of marker of the highly obstructed hematopoietic ability include a state that abnormal cells are observed in the peripheral blood or bone marrow. Presence or absence of abnormal cells can be evaluated by preparing a smear preparation from the peripheral blood or bone marrow aspirate obtained by, for example, blood collection or bone marrow aspiration, and observing the preparation under a microscope.

The thrombomodulin used for the present invention is known to have an action to (1) selectively bind to thrombin and (2) promote the activation of the protein C by thrombin. In addition, it is preferred that the thrombomodulin generally has (3) an action to extend the thrombin coagulation time, and/or (4) an action to suppress the platelet aggregation by thrombin. Such actions of thrombomodulin may be referred to as thrombomodulin activity. As for the thrombomodulin activity, thrombomodulin preferably has the actions of (1) and (2) mentioned above, and more preferably all the actions of (1) to (4) mentioned above.

As for the action to promote the activation of the protein C by thrombin, the activity of the action to promote the activation of the protein C, or the presence or absence of such an action can easily be confirmed by the test methods clearly described in various known publications including Japanese Patent Unexamined Publication No. 64-6219. The action to extend the thrombin coagulation time and/or the action to suppress the platelet aggregation caused by thrombin can also be easily confirmed in the same manner as mentioned above.

The thrombomodulin used for the present invention is not particularly limited so far as having thrombomodulin activity. The thrombomodulin is preferably a soluble thrombomodulin. The solubility of the soluble thrombomodulin in water such as distilled water used for injection (in the absence of a surfactant such as Triton X-100 or polidocanol, and generally around the neutral range) is preferably, for example, 1 mg/mL or more or 10 mg/mL or more; preferably 15 mg/mL or more or 17 mg/mL or more; more preferably 20 mg/mL or more, 25 mg/mL or more, or 30 mg/mL or more; particularly preferably 60 mg/mL or more. In some cases, the solubility is, for example, 80 mg/mL or more, or 100 mg/mL or more. For determining whether or not a soluble thrombomodulin is successfully dissolved in water, it is understood that clear appearance of a solution and the absence of apparently observable insoluble substances is served as simple criteria, after the soluble thrombomodulin is dissolved in water and the solution is observed by visual inspection, for example, just under a white light at a position corresponding to an illumination of approximately 1000 luxes. It is also possible to observe the presence or absence of any residue after filtration.

The thrombomodulin used for the present invention preferably comprises the amino acid sequence consisting of the amino acid residues at the positions 19 to 132 of SEQ ID NO: 1, which has been known as the central portion of the thrombomodulin activity of human thrombomodulin, and the thrombomodulin is not particularly limited, so long as the thrombomodulin comprises the amino acid sequence consisting of the amino acid residues at the positions 19 to 132 of SEQ ID NO: 1. The amino acid sequence consisting of the amino acid residues at the positions 19 to 132 of SEQ ID NO: 1 may be naturally or artificially mutated, so long as the sequence has an action to promote the activation of the protein C by thrombin, namely, the thrombomodulin activity. Specifically, the sequence may comprise substitution, deletion, or addition of one or more amino acid residue in the amino acid sequence consisting of the amino acid residues at the positions 19 to 132 of SEQ ID NO: 1. Acceptable level of the mutation is not particularly limited, so long as the amino acid sequence has the thrombomodulin activity. An example includes a homology 50% or more as amino acid sequences, and the homology is preferably 70% or more, more preferably 80% or more, further preferably 90% or more, particularly preferably 95% or more, and most preferably 98% or more. These amino acid sequences are referred to as homologous mutation sequences. As described later, the mutated amino acid sequences can be easily obtained by using ordinary gene manipulation techniques.

The amino acid sequence of SEQ ID NO: 3 comprises the mutation of Val as the amino acid at the position 125 of the sequence of SEQ ID NO: 1 to Ala. The thrombomodulin used for the present invention also preferably comprises the amino acid sequence from the position 19 to 132 of SEQ ID NO: 3.

As described above, although the thrombomodulin used for the present invention is not particularly limited so long that the thrombomodulin has at least the amino acid sequence from the position 19 to 132 of SEQ ID NO: 1 or 3, or a homologous mutation sequence thereof, and comprises at least a peptide sequence having the thrombomodulin activity, preferred examples of the thrombomodulin include a peptide consisting of the sequence from the position 19 to 132 or 17 to 132 in either of SEQ ID NO: 1 or SEQ ID NO: 3, and a peptide consisting of a homologous mutation sequence of the aforementioned sequence and having at least the thrombomodulin activity. A peptide consisting of the sequence from the position 19 to 132 in either of SEQ ID NO: 1 or SEQ ID NO: 3 is more preferred. In another embodiment, a peptide consisting of a homologous mutation sequence of the sequence from the position 19 to 132 or 17 to 132 in either of SEQ ID NO: 1 or SEQ ID NO: 3 and having at least the thrombomodulin activity is more preferred.

As another embodiment of the thrombomodulin according to the present invention, the thrombomodulin preferably comprises the amino acid sequence from the positions 19 to 480 of SEQ ID NO: 5, which is not particularly limited so long as the thrombomodulin comprises the amino acid sequence from the position 19 to 480 of SEQ ID NO: 5. The amino acid sequence from the position 19 to 480 of SEQ ID NO: 5 may be a homologous mutation sequence thereof, so long as the sequence has an action to promote the activation of the protein C by thrombin, i.e., the thrombomodulin activity.

The sequence of SEQ ID NO: 7 comprises the mutation of Val as the amino acid at the position 473 of the sequence of SEQ ID NO: 5 to Ala. The thrombomodulin used for the present invention also preferably comprises the amino acid sequence from the position 19 to 480 of SEQ ID NO: 7.

As described above, although the thrombomodulin used for the present invention is not particularly limited so long as the thrombomodulin has at least the sequence from the position 19 to 480 in either of SEQ ID NO: 5 or SEQ ID NO: 7, or a homologous mutation sequence thereof, and comprises at least a peptide sequence having the thrombomodulin activity, preferred examples of the thrombomodulin include a peptide consisting of the sequence from the position 19 to 480 or 17 to 480 in either of SEQ ID NO: 5 or SEQ ID NO: 7, and a peptide consisting of a homologous mutation sequence of the aforementioned sequence and having at least the thrombomodulin activity. A peptide consisting of the sequence from the position 19 to 480 of SEQ ID NO: 5 or 7 is more preferred. In another embodiment, a peptide consisting of a homologous mutation sequence of the sequence from the position 19 to 480 or 17 to 480 in either of SEQ ID NO: 5 or SEQ ID NO: 7, and having the thrombomodulin activity is more preferred.

As another embodiment of the thrombomodulin according to the present invention, the thrombomodulin preferably comprises the amino acid sequence from the position 19 to 515 of SEQ ID NO: 9, which is not particularly limited so long as the thrombomodulin comprises the amino acid sequence from the position 19 to 515 of SEQ ID NO: 9. The amino acid sequence from the position 19 to 515 of SEQ ID NO: 9 may be a homologous mutation sequence thereof, so long as the sequence has an action to promote the activation of the protein C by thrombin, i.e., the thrombomodulin activity.

The amino acid sequence of SEQ ID NO: 11 comprises the mutation of Val as the amino acid at the position 473 of SEQ ID NO: 9 to Ala. The thrombomodulin used for the present invention also preferably comprises the amino acid sequence from the position 19 to 515 of SEQ ID NO: 11.

As described above, although the thrombomodulin used for the present invention is not particularly limited so long as the thrombomodulin has at least the sequence from the position 19 to 515 in either of SEQ ID NO: 9 or SEQ ID NO: 11, or a homologous mutation sequence thereof, and comprises a peptide sequence having at least the thrombomodulin activity, more preferred examples include a peptide having the sequence from position 19 to 516, 19 to 515, 17 to 516, or 17 to 515 in either of SEQ ID NO: 9 or SEQ ID NO: 11, and a peptide consisting of a homologous mutation sequence of the aforementioned sequence and having at least the thrombomodulin activity. A peptide having the sequence from the position 19 to 516, 19 to 515, 17 to 516, or 17 to 515 of SEQ ID NO: 9 is particularly preferred. A mixture thereof is also a preferred example. In another embodiment, a peptide having the sequence from the position 19 to 516, 19 to 515, 17 to 516, or 17 to 515 of SEQ ID NO: 11 is particularly preferred. A mixture thereof is also a preferred example. Further, a peptide consisting of a homologous mutation sequence thereof and having at least the thrombomodulin activity is also a preferred example.

A peptide having a homologous mutation sequence is as described above, and means a peptide that may comprise substitution, deletion, or addition of at least one, namely, one or more, preferably several (for example, 1 to 20, preferably 1 to 10, more preferably 1 to 5, particularly preferably 1 to 3) amino acid residues, in the amino acid sequence of the subjected peptide. Although acceptable level of mutation is not particularly limited so long as the peptide has the thrombomodulin activity, an example of the acceptable level of homology includes 50% or more as an amino acid sequences, and the homology may be preferably 70% or more, more preferably 80% or more, further preferably 90% or more, particularly preferably 95% or more, and most preferably 98% or more.

Preferred examples of the thrombomodulin used for the present invention also include the peptide consisting of the sequence of SEQ ID NO: 14 (462 amino acid residues), the peptide consisting of the sequence of SEQ ID NO: 8 (272 amino acid residues), and the peptide consisting of the sequence of SEQ ID NO: 6 (236 amino acid residues) described in Japanese Patent Unexamined Publication No. 64-6219.

Although the thrombomodulin used for the present invention is not particularly limited so long as the thrombomodulin has at least the amino acid sequence from the position 19 to 132 in either of SEQ ID NO: 1 or SEQ ID NO: 3. Among them, a peptide having at least the amino acid sequence from the position 19 to 480 in either of SEQ ID NO: 5 or SEQ ID NO: 7 is preferred, and a peptide having at least the amino acid sequence from the position 19 to 515 in either of SEQ ID NO: 9 or SEQ ID NO: 11 is more preferred. A more preferred example of the peptide having at least the amino acid sequence from the position 19 to 515 in either of SEQ ID NO: 9 or SEQ ID NO: 11 is a peptide having the sequence from the position 19 to 516, 19 to 515, 17 to 516, or 17 to 515 in either of SEQ ID NO: 9 or SEQ ID NO: 11. Furthermore, a mixture of peptides each consisting of the sequence from the position 19 to 516, 19 to 515, 17 to 516, or 17 to 515 in either of SEQ ID NO: 9 or SEQ ID NO: 11 is also a preferred example.

In the case of the aforementioned mixture, an example of a mixing ratio of a peptide that starts from the position 17 in either of SEQ ID NO: 9 or SEQ ID NO: 11 and a peptide that starts from the position 19 in either of SEQ ID NO: 9 or SEQ ID NO: 11 is 30:70 to 50:50. A preferred example includes the ratio of 35:65 to 45:55.

An example of a mixing ratio of a peptide that terminates at the position 515 in either of SEQ ID NO: 9 or SEQ ID NO: 11 and a peptide that terminates at the position 516 is 70:30 to 90:10. A preferred example include the ratio of 75:25 to 85:15.

The mixing ratio of the peptides can be determined by an ordinary method.

The sequence from the position 19 to 132 of SEQ ID NO: 1 corresponds to the sequence from the position 367 to 480 of SEQ ID NO: 9, and the sequence from the position 19 to 480 of SEQ ID NO: 5 corresponds to the sequence from the position 19 to 480 of SEQ ID NO: 9. Further, the sequence from the position 19 to 132 of SEQ ID NO: 3 corresponds to the sequence from the position 367 to 480 of SEQ ID NO: 11, and the sequence from the position 19 to 480 of SEQ ID NO: 7 corresponds to the sequence from the position 19 to 480 of SEQ ID NO: 11. Furthermore, the sequences from the position 1 to 18 of SEQ ID NOS: 1, 3, 5, 7, 9, and 11 are all identical to one another.

As described below, these thrombomodulins according to the present invention can be obtained from transformant cells prepared by transfecting host cells with a DNA encoding the peptide (specifically, the nucleotide sequences of SEQ ID NOS: 2, 4, 6, 8, 10, 12, and the like) by using a vector.

It is sufficient that these peptides only have the aforementioned amino acid sequences, and a sugar chain may be attached or not attached, which not particularly limited. In gene manipulation techniques, a type of a sugar chain, a position to which a sugar chain is added, and a level of addition thereof differ depending on a type of host cells used, and any techniques may be used. As for binding position of a sugar chain and a type thereof, facts described in Japanese Patent Unexamined Publication No. 11-341990 are known, and the thrombomodulins according to the present invention may be added with the same sugar chain at the same position. As described later, although the method is not limited to gene manipulation techniques, when the thrombomodulin is obtained by a gene manipulation technique, a nucleotide sequence encoding the amino acid sequence from the position 1 to 18 of SEQ ID NO: 9, a nucleotide sequence encoding the amino acid sequence from the position 1 to 16 of SEQ ID NO: 9, and other known signal sequences such as the signal sequence of a human tissue plasminogen activator can be used as a signal sequence usable for expression (International Patent Publication WO88/9811).

When a DNA sequence encoding thrombomodulin is introduced into a host cell, an example of preferred methods include a method of incorporating a DNA sequence encoding thrombomodulin into, preferably, a vector, more preferably an expression vector capable of being expressed in animal cells, and then introducing the DNA with the vector. An expression vector is a DNA molecule that is constituted with a promoter sequence, a sequence for adding a ribosome binding site to mRNA, a DNA sequence encoding a protein to be expressed, a splicing signal, a terminator sequence for transcription termination, a replication origin sequence, and the like. Examples of preferred animal cell expression vector include pSV2-X reported by Mulligan R. C. et al. (Proc. Natl. Acad. Sci. U.S.A., 78, 2072, 1981); pBP69T (69-6) reported by Howley P. M. et al. (Methods in Emzymology, 101, 387, 1983, Academic Press), and the like.

Examples of host cells that can be used in the production of these peptides include Chinese hamster ovary (CHO) cells, COS-1 cells, COS-7 cells, VERO (ATCC CCL-81) cells, BHK cells, canine kidney-derived MDCK cells, hamster AV-12-664 cells, and the like. Examples of host cell derived from human include HeLa cells, WI38 cells, and human 293 cells. CHO cells are preferred, since they are very common, and among CHO cells, DHFR-CHO cells are more preferred.

In the process of gene manipulation or process of producing a peptide, microorganisms such as Escherichia coli are frequently used, and a host-vector system suitable for each microorganism is preferably used. An appropriate vector system can also be selected for the aforementioned host cells. A thrombomodulin gene used in a genetic recombination technique has been cloned, and examples of production of thrombomodulin by a genetic recombination technique have been disclosed. Further, methods for obtaining a purified product thereof are also known (Japanese Patent Unexamined Publication Nos. 64-6219, 2-255699, 5-213998, 5-310787, 7-155176; and J. Biol. Chem., 264: 10351-10353, 1989). Therefore, the thrombomodulin used for the present invention can be produced by the methods described in the aforementioned reports, or methods similar thereto. For example, Japanese Patent Unexamined Publication No. 64-6219 discloses the Escherichia coli K-12 strain DH5 (ATCC Accession No. 67283) comprising a plasmid pSV2TMJ2 containing a DNA encoding the full-length thrombomodulin. A strain deposited at the National Institute of Bioscience and Human-Technology (currently, the National Institute of Advanced Industrial Science and Technology, International Patent Organism Depositary), Escherichia coli DH5/pSV2TM J2 (FERM BP-5570) can also be used. The thrombomodulin of the present invention can be prepared by a known gene manipulation technique using a DNA encoding the full-length thrombomodulin as a starting material.

The thrombomodulin used for the present invention may be prepared by a conventionally known method or a similar method. For example, the thrombomodulin can be prepared by referring to the aforementioned method of Yamamoto et al. (Japanese Patent Unexamined Publication No. 64-6219) or the method described in Japanese Patent Unexamined Publication No. 5-213998. Specifically, a human-derived thrombomodulin gene may be made into, for example, a DNA encoding the amino acid sequence of SEQ ID NO: 9 by a gene manipulation technique, and may be further modified as required. For such modification, for example, in order to prepare a DNA encoding the amino acid sequence of SEQ ID NO: 11 (specifically, consisting of the nucleotide sequence of SEQ ID NO: 12), site-directed mutagenesis is performed for the codon encoding the amino acid residue at the position 473 of SEQ ID NO: 9 (in particular, the nucleotide at the position 1418) according to the method described by Zoller M. J. et al. (Method in Enzymology, 100:468-500, 1983, Academic Press). For example, by using a synthetic DNA for mutation having the nucleotide sequence of SEQ ID NO: 13, a DNA corresponding to the sequence of SEQ ID NO: 10 where the nucleotide T at the position 1418 is converted to the nucleotide C may be prepared.

A DNA prepared as described above is incorporated into, for example, Chinese hamster ovary (CHO) cells to obtain transformant cells, and the cells are subjected to appropriate selection, and then purified thrombomodulin can be prepared by a known method from the culture obtained by culturing the cells. As described above, it is preferable to transfect the host cells as mentioned above with the DNA encoding the amino acid sequence of SEQ ID NO: 9 (SEQ ID NO: 10). The method for producing thrombomodulin used for the present invention is not limited to the aforementioned method. For example, it is also possible to extract and purify the thrombomodulin from urine, blood, other body fluids and the like, or extract and purify the thrombomodulin from a tissue producing thrombomodulin or a culture of the aforementioned tissue and the like Further, the thrombomodulin may be further subjected to a cleavage treatment using a protease, as required.

When thrombomodulin used in the present invention is produced by the aforementioned cell culture method, the N-terminal amino acid may sometimes be found to be diversified as a result of posttranslational modification of the protein. For example, the amino acid residue at the position 17, 18, 19, or 22 of SEQ ID NO: 9 may sometimes constitute the N-terminus. Further, the N-terminal amino acid residue may sometimes be modified such that the glutamic acid at the position 22 is converted to pyroglutamic acid. It is preferred that the amino acid residue at the position 17 or 19 constitutes the N-terminus, and it is more preferred that the amino acid residue at the position 19 constitutes the N-terminus In another embodiment, it is preferred that the amino acid at the position 17 constitutes the N-terminus. As for the aforementioned modification, diversification and the like, the same examples are applied for the sequence of SEQ ID NO: 11.

Further, when thrombomodulin is produced by using a DNA having the nucleotide sequence of SEQ ID NO: 10, the C-terminal amino acid may be diversified, and as a result, a shorter peptide by one amino acid may be produced. More specifically, the C-terminal amino acid residue may sometimes be modified so that, for example, the amino acid residue at the position 515 constitutes the C-terminus, and further subjected to amide formation. Therefore, a peptide having diversified N-terminal amino acid and C-terminal amino acid or a mixture thereof may sometimes be produced. It is preferred that the amino acid at the position 515 constitutes the C-terminus. In another embodiment, it is preferred that the amino acid at the position 516 constitutes the C-terminus. As for the aforementioned modification, diversification and the like, the examples are applied to a DNA having the nucleotide sequence of SEQ ID NO: 12.

The thrombomodulin obtained by the aforementioned method may sometimes be a mixture of peptides having diversified N-termini and C-termini. A specific example includes a mixture of peptides having the sequences from the position 19 to 516, 19 to 515, 17 to 516, and 17 to 515 of SEQ ID NO: 9.

Then, isolation and purification of thrombomodulin from a culture supernatant or culture obtained as described above can be carried out by known methods [edited by Takeichi Horio, Tanpakushitsu/Koso no Kiso Jikken Ho (Fundamental Experimental Methods for Proteins and Enzymes)]. For example, it is preferable to use ion exchange chromatography or adsorption chromatography, which utilizes an interaction between thrombomodulin and a chromatographic carrier on which functional groups having a charge opposite to that of thrombomodulin are immobilized. Another preferred example is affinity chromatography utilizing specific affinity with thrombomodulin. Preferred examples of adsorbent include thrombin that is a ligand of thrombomodulin and an anti-thrombomodulin antibody. As the antibody, anti-thrombomodulin antibodies having appropriate properties or recognizing appropriate epitopes can be used. Examples include, for example, those described in Japanese Patent Publication (Kokoku) No. 5-42920, Japanese Patent Unexamined Publication Nos. 64-45398 and 6-205692 and the like. Other examples include gel filtration chromatography and ultrafiltration, which utilize the molecular size of thrombomodulin. Other examples further include hydrophobic chromatography that utilizes hydrophobic bond between a chromatographic carrier on which hydrophobic groups are immobilized, and a hydrophobic portion of thrombomodulin. Furthermore, hydroxyapatite may be used as a carrier in adsorption chromatography, of which examples include, for example, those described in Japanese Patent Unexamined Publication No. 9-110900. These means may be used in combination, as required. Although degree of purification can be selected depending on a purpose of use and the like, it is desirable to purify thrombomodulin until a single band is obtained as a result of electrophoresis, preferably SDS-PAGE, or a single peak is obtained as a result of gel filtration HPLC or reverse phase HPLC of the isolated and purified product. It should be understood that, when two or more types of thrombomodulins are used, it is preferred that only the bands of the thrombomodulins are substantially obtained, and it is not required to obtain one single band.

Specific examples of the purification method used in the present invention include a purification method using the thrombomodulin activity as a criterion, for example, a purification method comprising roughly purifying a culture supernatant or a culture product with an ion exchange column Q-Sepharose Fast Flow to collect a fraction having the thrombomodulin activity; then purifying the fraction with an affinity column, DIP-thrombin-agarose (diisopropylphosphorylthrombin agarose) column, as the main purification step to recover a fraction having a strong thrombomodulin activity; then concentrating the recovered fraction and followed by gel filtration to obtain a thrombomodulin active fraction as a purified product (Gomi K. et al., Blood, 75: 1396-1399, 1990). An example of the thrombomodulin activity used as the criterion is an activity of promoting the activation of the protein C by thrombin. Other preferred examples of the purification method will be exemplified below.

An appropriate ion exchange resin having good adsorptive condition for thrombomodulin is selected and purification by ion exchange chromatography is performed. A particularly preferred example includes a method comprising the use of Q-Sepharose Fast Flow equilibrated with a 0.02 mol/L Tris-HCl buffer (pH 7.4) containing 0.18 mol/L NaCl. After washing as required, elution can be performed with a 0.02 mol/L Tris-HCl buffer (pH 7.4) containing 0.3 mol/L NaCl, for example, to obtain thrombomodulin as a roughly purified product.

Then, for example, a substance having specific affinity to thrombomodulin can be immobilized on a resin to perform purification by affinity chromatography. Preferred examples include a DIP-thrombin-agarose column and an anti-thrombomodulin monoclonal antibody column. In the case of the DIP-thrombin-agarose column, the column is equilibrated beforehand with a 20 mmol/L Tris-HCl buffer (pH 7.4) containing 100 mmol/L NaCl and 0.5 mmol/L calcium chloride, and the aforementioned roughly purified product is then charged on the column, washed as required, and then eluted with, for example, a 20 mmol/L Tris-HCl buffer (pH 7.4) containing 1.0 mol/L NaCl and 0.5 mmol/L calcium chloride to obtain thrombomodulin as a purified product. In the case of the anti-thrombomodulin monoclonal antibody column, an example of the method comprises: contacting an anti-thrombomodulin monoclonal antibody solution in a 0.1 mol/L NaHCO₃ buffer (pH 8.3) containing 0.5 mol/L NaCl with Sepharose 4FF (GE Health Care Biosciences) activated with CNBr beforehand, subjecting a column filled with the resin consisting of Sepharose 4FF coupled with the anti-thrombomodulin monoclonal antibodies to be equilibrated beforehand with, for example, a 20 mmol/L phosphate buffer (pH 7.3) containing 0.3 mol/L NaCl, washing as required, and then eluting a product with a 100 mmol/L glycine-HCl buffer (pH 3.0) containing 0.3 mol/L NaCl. An effluent may be neutralised with an appropriate buffer to obtain a product as a purified product.

Subsequently, the purified product is adjusted to pH 3.5, and then charged on a cation exchanger, preferably SP-Sepharose FF (GE Health Care Biosciences) as a strong cation exchanger, equilibrated with a 100 mmol/L glycine-HCl buffer (pH 3.5) containing 0.3 mol/L NaCl, and washing is performed with the same buffer to obtain a non-adsorptive fraction. The resulting fraction is neutralized with an appropriate buffer to obtain a highly purified product. These product are preferably concentrated by ultrafiltration.

Further, it is also preferable to exchange the buffer by gel filtration. For example, a highly purified product concentrated by ultrafiltration can be charged on a Sephacryl S-300 column or 5-200 column equilibrated with a 20 mmol/L phosphate buffer (pH 7.3) containing 50 mmol/L NaCl, and then developed for fractionation with a 20 mmol/L phosphate buffer (pH 7.3) containing 50 mmol/L NaCl. The activity for promoting the activation of the protein C by thrombin can be confirmed to collect an active fraction and thereby obtain a buffer-exchanged highly purified product. In order to improve safety, a highly purified product obtained as described above is preferably filtered through an appropriate filter for eliminating viruses such as Planova 15N (Asahi Kasei Medical Co., Ltd.), and then the resultant can be concentrated by ultrafiltration to a desired concentration. Finally, the product is preferably filtered through an aseptic filtration filter.

With the medicament of the present invention, a pain associated with hematopoietic cell transplantation can be prevented and/or treated. The pain associated with hematopoietic cell transplantation is not particularly limited, so long as the pain is developed with hematopoietic cell transplantation. Examples include a pain developed with graft versus host disease (GVHD), sinusoidal obstruction syndrome (SOS), or transplantation-associated microangiopathy (TAM), and a pain developed with SOS is preferred. There is another embodiment in which a pain developed with GVHD is preferred.

GVHD can be typologically classified or diagnosed according to the GVHD guideline of the Japan Society for Hematopoietic Cell Transplantation (Non-patent document 1).

SOS can be diagnosed according to any one of such criteria as the Seattle criteria (McDonald G. B. et al., Hepatol., 4:116-22, 1984), the modified Seattle criteria (Shulman H. M. et al., Bone Marrow Transplant, 10:197-214, 1992), and the Baltimore criteria (Jones R. J. et al., Transplant, 44:778-83, 1987).

TAM can be diagnosed according to the criteria of BMT-CTN in North America (Ho V. T. et al., Biol. Blood Marrow Transplant, 11:571-5, 2005) or EBMT-ELN in Europe (Ruutu T. et al., Haematologica, 92:95-100, 2007).

As the method for evaluating a pain, there can be used a method of diagnosing the presence or absence of pain, and when it is desired to quantitatively evaluate a pain, there can be used the visual analogue scale (VAS), verbal rating scale (VRS) or numerical rating scale (NRS) (Williamson, J. Clin. Nurs. 5, 14(7):798-804, 2000).

The region at which the pain is developed with hematopoietic cell transplantation is not particularly limited so long that a pain is developed with hematopoietic cell transplantation. Examples include at least one region selected from the group consisting of the eyeball, oral cavity, abdomen and hypochondrium, and the hypochondrium is preferred. As for type of the pain developed with hematopoietic cell transplantation, examples of the pain include acute pain, chronic pain, visceral pain, somatic pain, parietal pain, referred pain, radiating pain, ischemic pain, tenderness, colick, tingling, and the like, but it is not particularly limited, so long as it is a pain associated with hematopoietic cell transplantation.

One embodiment of the present invention is preferred from a viewpoint of fast-acting property of the pain improving effect. Although the time required for disappearance of pain is not particularly limited so long that, when coagulopathy or weight gain is developed, the pain disappears before improvement of these symptoms. An example is disappearance of a pain in 2 days after the administration of thrombomodulin. The period of time is preferably 36 hours, more preferably 24 hours, still more preferably 12 hours, particularly preferably 6 hours, and most preferably 4 hours.

According to one embodiment of the medicament of the present invention, by administering the medicament to a hematological malignancy patient who is to undergo hematopoietic cell transplantation, or a hematological malignancy patient who has undergone hematopoietic cell transplantation, hematopoietic activity can be improved at the time of hematopoietic cell transplantation.

Degree of the improvement of hematopoietic activity can be determined by measuring blood count of peripheral blood. The peripheral blood count can be measured with a measurement apparatus using the electric resistance method or the optical measurement method, or the electric resistance method and the optical measurement method. Measurement according to a method of using a hemacytometer is also available.

According to one embodiment of the medicament of the present invention, by administering the medicament to a hematological malignancy patient who is to undergo hematopoietic cell transplantation, or a hematological malignancy patient who has undergone hematopoietic cell transplantation, the hematological malignancy can be treated and/or improved.

According to one embodiment of the medicament of the present invention, by administering it to a hematological malignancy patient who is to undergo hematopoietic cell transplantation, or a hematological malignancy patient who has undergone hematopoietic cell transplantation, therapeutic and/or improving effect of hematopoietic cell transplantation on a hematological malignancy can be augmented.

Degree of cure and/or improvement of a hematological malignancy can be observed by 1) measuring number of abnormal cells in the bone marrow. In the case of a hematological malignancy for which abnormality of the genes is found, the degree can be determined by appropriately using the fluorescence in situ hybridization (FISH) method or the polymerase chain reaction (PCR) method in combination.

Form of the medicament of the present invention is not particularly limited, so long as the medicament contains thrombomodulin as an active ingredient, and medicaments used for a hematopoietic cell transplantation patient can also be prepared as a single or multiple preparations and used as required. Specifically, the present invention provides a medicament for prophylactic and/or therapeutic treatment of a pain associated with hematopoietic cell transplantation, a medicament for improving hematopoietic activity at the time of hematopoietic cell transplantation, a medicament for therapeutic treatment and/or improvement of a hematological malignancy, and a medicament for augmenting therapeutic and/or improving effect of hematopoietic cell transplantation on a hematological malignancy, each of which comprises thrombomodulin and at least one therapeutic agent selected from the group consisting of an immunosuppressant and a therapeutic agent for hematological malignancy in combination. As the medicament comprising thrombomodulin and at least one therapeutic agent selected from the group consisting of an immunosuppressant and a therapeutic agent for hematological malignancy in combination, a medicament comprising thrombomodulin and an immunosuppressant in combination is preferred. Another embodiment is also preferred wherein a medicament consists of thrombomodulin, an immunosuppressant, and a therapeutic agent for hematological malignancy in combination.

The present invention also provides a medicament for prophylactic and/or therapeutic treatment of a pain associated with hematopoietic cell transplantation, a medicament for improving hematopoietic activity at the time of hematopoietic cell transplantation, a medicament for therapeutic treatment and/or improvement of a hematological malignancy, and a medicament for augmenting therapeutic effect and/or improving effect of hematopoietic cell transplantation on a hematological malignancy, each of which comprises thrombomodulin and at least one therapeutic agent selected from the group consisting of an immunosuppressant, a therapeutic agent for hematological malignancy, and a prophylactic and/or therapeutic agent for a complication associated with hematopoietic cell transplantation in combination.

Examples of the aforementioned immunosuppressant include corticosteroids, alkaloid agents, calcineurin inhibitors, cyclin-dependent kinase inhibitors, antithymocyte globulin, antilymphocyte globulin, anti-cytokine antibodies, anti-CD antibodies, and the like. Examples of the agents include, but not limited to, predonisolone, methylprednisolone, hydrocortisone, triamcinolone, betamethasone, dexamethasone, cyclosporin, tacrolimus, sirolimus, rapamycin, Atgam, muromonab-CD3, basiliximab, daclizumab, infliximab, etanercept, adalimumab, tocilizumab, and gusperimus, and tacrolimus, sirolimus, rapamycin, and cyclosporin are preferred. Preferred examples also include use of the antimetabolites and alkylating agents mentioned later as the immunosuppressant. Methotrexate, cytarabine, and mycophenolic acid are also preferred, however, the immunosuppressant is not limited to these examples.

The therapeutic agent for hematological malignancy is a medicament used for therapeutic treatment of hematological malignancy which includes chemotherapeutic agents. Examples include DNA or RNA polymerase inhibitors, antimetabolites, anti-tumor enzymes, alkylating agents, microtubule polymerization inhibitors, hormone therapy agents, molecular targeting agents and the like. Examples of such agents include doxorubicin, daunorubicin, idarubicin, aclarubicin, ethoposide, methotrexate, cytarabine, cladribine, mycophenolic acid, mizoribine, azathioprine, mitomycin C, L-asparaginase, cyclophosphamide, busulfan, melphalan, vincristine, vinblastine, filgrastim, lenograstim, nartograstim, tretinoin (all-trans retinoic acid, ATRA), rituximab, ofatumumab, alemtuzimab, gemutuzumab ozogamicin, 131-I tositumomab, 90-Y ibritumomab tiuxetan, daclizumab, IDEC-114, and KW-0761, but not limited to these examples. These agents are used independently or as an appropriate combination according to disease type of the hematological malignancy.

Examples of the aforementioned prophylactic and/or therapeutic agent for a complication associated with hematopoietic cell transplantation include steroids, mycophenolic acid, antithymocyte globulin, heparin, prostaglandin E1, ursodeoxycholic acid, frozen fresh plasma, tissue plasminogen activator, defibrotide, and the like, and mycophenolic acid, prostaglandin E1, ursodeoxycholic acid, and defibrotide are preferred, but not limited to these examples.

The medicament of the present invention comprising thrombomodulin and at least one therapeutic agent selected from the group consisting of an immunosuppressant and a therapeutic agent for hematological malignancy in combination may be in the form of a formulation consisting of one dosage form containing all the ingredients as a mixture, or in the form of a combination of the ingredients not mixed and independently administrable from plural containers.

As the conditioning regimen of the hematopoietic cell transplantation used for the present invention, in addition to use of the aforementioned therapeutic agent for hematological malignancy, radiotherapy is optionally carried out in combination. Examples of irradiation method of the radiotherapy include the source axis distance (SAD) method, source skin distance (SSD) method, sweeping beam method, moving beam method, and moving couch method. Examples of irradiation schedule of TBI include the single irradiation method and multiple irradiation method. Examples of dosage include 10 to 12 Gy in the case of the single irradiation method, as well as 2 Gy per one irradiation and 12 Gy in six days, 2.25 Gy per one irradiation and 15.75 Gy in 7 days, twice or three times of 1.8 Gy per day and about 15 Gy in total, once or twice of 2 to 3 Gy per day and 12 Gy in total, and the like. However, the method and the dosage are not limited to those mentioned above. Dosage rate for TBI is, for example, 5 to 15 cGy/min. Further, examples of the conditioning regimen for a case without the radiotherapy, called as mini-transplantation, include use of an appropriate combination of fludarabine, cladribine, and antithymocyte globulin, but not limited to these examples.

As the medicament of the present invention comprising thrombomodulin and at least one therapeutic agent selected from the group consisting of an immunosuppressant and a therapeutic agent for hematological malignancy in combination, thrombomodulin and at least one active ingredient selected from the group consisting of an immunosuppressant and a therapeutic agent for hematological malignancy can be simultaneously administered as a complete mixture thereof. Thrombomodulin and at least one therapeutic agent selected from the group consisting of an immunosuppressant and a therapeutic agent for hematological malignancy of the medicament of the present invention can be administered simultaneously or successively as separate active ingredients.

In the case of successively administering the ingredients of the medicament comprising thrombomodulin and an immunosuppressant in combination, for example, the order of administration of thrombomodulin and the immunosuppressant is not limited. The immunosuppressant is preferably administered after thrombomodulin. There is another preferred embodiment in which thrombomodulin is administered after the immunosuppressant. Although thrombomodulin can be administered before hematopoietic cell transplantation, after hematopoietic cell transplantation, or during hematopoietic cell transplantation, thrombomodulin is preferably administered before hematopoietic cell transplantation or after hematopoietic cell transplantation, and the thrombomodulin is more preferably administered after hematopoietic cell transplantation. There is another embodiment in which the thrombomodulin is preferably administered before hematopoietic cell transplantation. Furthermore, the thrombomodulin may be preferably administered during hematopoietic cell transplantation. The immunosuppressant is preferably administered during hematopoietic cell transplantation or after hematopoietic cell transplantation. The order of administration or site of administration may be appropriately changed according to conditions of an objective patient.

By administering the medicament of the present invention together with at least one therapeutic agent selected from the group consisting of an immunosuppressant and a therapeutic agent for hematological malignancy, a pain associated with hematopoietic cell transplantation can be prevented and/or treated. Examples of the immunosuppressant include the agents exemplified above as immunosuppressants, and examples of the therapeutic agent for hematological malignancy include the agents exemplified above as therapeutic agents for hematological malignancy.

Specifically, the medicament of the present invention can be administered together with an immunosuppressant. Further, the medicament of the present invention can be administered together with a therapeutic agent for hematological malignancy. Furthermore, the medicament of the present invention can be administered together with an immunosuppressant and a therapeutic agent for hematological malignancy.

In the case of successively administering ingredients of the medicament comprising thrombomodulin, an immunosuppressant, and a therapeutic agent for hematological malignancy in combination, for example, the order of administration of the therapeutic agent for hematological malignancy is not particularly limited, so long as the therapeutic agent for hematological malignancy is administered before hematopoietic cell transplantation or after hematopoietic cell transplantation. The therapeutic agent for hematological malignancy is preferably administered after administration of thrombomodulin and the immunosuppressant. There is another embodiment in which thrombomodulin and the immunosuppressant are preferably administered after administration of the therapeutic agent for hematological malignancy. Although the therapeutic agent for hematological malignancy can also be administered before hematopoietic cell transplantation, after hematopoietic cell transplantation, or during hematopoietic cell transplantation, the agent is preferably administered before hematopoietic cell transplantation or after hematopoietic cell transplantation. Order of the administration or administration site can be appropriately changed according to conditions of an objective patient.

Further, in the case of successively administering ingredients of the medicament comprising thrombomodulin, an immunosuppressant, a therapeutic agent for hematological malignancy, and a prophylactic and/or therapeutic agent for a complication associated with hematopoietic cell transplantation in combination, for example, although the order of administration of the prophylactic and/or therapeutic agent for a complication associated with hematopoietic cell transplantation is not particularly limited so long as the prophylactic and/or therapeutic agent for a complication associated with hematopoietic cell transplantation is administered before hematopoietic cell transplantation or after hematopoietic cell transplantation. The prophylactic and/or therapeutic agent for a complication associated with hematopoietic cell transplantation is preferably administered after administration of thrombomodulin, the immunosuppressant, and the therapeutic agent for hematological malignancy. There is another embodiment in which thrombomodulin, the immunosuppressant, and the therapeutic agent for hematological malignancy are preferably administered after administration of the prophylactic and/or therapeutic agent for a complication associated with hematopoietic cell transplantation. Although the prophylactic and/or therapeutic agent of for a complication associated with hematopoietic cell transplantation can also be administered before hematopoietic cell transplantation, after hematopoietic cell transplantation, or during hematopoietic cell transplantation, the agent is preferably administered after hematopoietic cell transplantation. Order of the administration or administration site can be appropriately changed according to conditions of an objective patient.

The medicament of the present invention may contain a carrier. As the carrier usable in the present invention, a water-soluble carrier is preferred, and the medicament of the present invention can be prepared by adding, for example, sucrose, glycerol and the like, as well as a pH modifier consisting of a mineral salt, and the like as additives. Further, if necessary, amino acids, salts, carbohydrates, surfactants, albumin, gelatin and the like may be added as disclosed in Japanese Patent Unexamined Publication Nos. 64-6219 and 6-321805. Addition of a preservative is also preferred. Preferred examples include paraoxybenzoic acid esters, and particularly preferred examples include methyl paraoxybenzoate. Amount of the preservative to be added is usually, for example, from 0.01 to 1.0% (percent by weight, the same shall apply to the following descriptions), preferably from 0.1 to 0.3%. Method for adding these additives is not particularly limited. However, in the case of preparing a lyophilized product, examples include, for example, a method of mixing a solution containing at least one therapeutic agent selected from an immunosuppressant and a therapeutic agent for hematological malignancy, and a solution containing thrombomodulin, then adding additives to the mixture, and mixing the resulting mixture, and a method of mixing additives with at least one therapeutic agent selected from an immunosuppressant and a therapeutic agent for hematological malignancy dissolved in water, water for injection, or an appropriate buffer beforehand, adding a solution containing thrombomodulin to the mixture, mixing the resulting mixture to prepare a solution, and lyophilizing the solution, in manners as those commonly employed. When the medicament of the present invention is a medicament comprising a combination of the components of the medicament, each component is preferably prepared by adding a carrier according to an appropriate preparation method. The medicament of the present invention may be provided in the form of an injection, or in the form of a lyophilized preparation to be dissolved upon use.

Examples of the method for preparing the medicament include a method of filling a solution containing 0.05 to 15 mg, preferably 0.1 to 5 mg, of thrombomodulin in 1 mL of water, water for injection, or an appropriate buffer, any one of an anti-platelet agent, an anticoagulant, and a thrombolytic agent, and the aforementioned additives in a volume of, for example, 0.5 to 10 mL in an ampoule or vial, then freezing the solution, and drying the frozen solution under reduced pressure. The solution, per se, may be used as a preparation for injection consisting of aqueous solution.

The medicament of the present invention is desirably administered by parenteral administration such as intravenous administration, intramuscular administration, and subcutaneous administration. The medicament may also be administered by oral administration, intrarectal administration, intranasal administration, sublingual administration or the like. When the medicament of the present invention is a medicament comprising a combination of multiple active ingredients, each active ingredient of the medicament is preferably administered by an administration method suitable for the ingredient.

Examples of method for the intravenous administration include a method of administering a desired close of the medicament at one time, and intravenous administration by drip infusion.

The method of administering a desired dose of the medicament at one time is preferred from the viewpoint that the method requires only a short period of administration time. When the medicament is administered at one time, a period required for administration by using an injectable syringe may generally varies. In general, the period of time required for the administration is preferably 2 minutes or shorter, more preferably 1 minute or shorter, still more preferably 30 seconds or shorter, although it depends on a volume to be administered. Although the minimum administration time is not particularly limited, the period is preferably 1 second or longer, more preferably 5 seconds or longer, still more preferably 10 seconds or longer. The dose is not particularly limited so long that the close is within the aforementioned preferred dose. Intravenous administration by drip infusion is also preferred from a viewpoint that blood level of thrombomodulin can be easily kept constant.

A daily dose of the medicament of the present invention may vary depending on age, body weight of patients, severity of disease, administration route and the like. In general, the maximum dose is preferably 20 mg/kg or less, more preferably 10 mg/kg or less, still more preferably 5 mg/kg or less, particularly preferably 2 mg/kg or less, and most preferably 1 mg/kg or less, and the minimum dose is preferably 0.001 mg/kg or more, more preferably 0.005 mg/kg or more, still more preferably 0.01 mg/kg or more, particularly preferably 0.02 mg/kg or more, and most preferably 0.05 mg/kg or more, in terms of the amount of thrombomodulin.

In the case of intravenous administration by drip infusion, although the dose is not particularly limited so long as the dose is within the aforementioned preferred dose, the maximum daily dose is preferably 1 mg/kg or less, more preferably 0.5 mg/kg or less, still more preferably 0.1 mg/kg or less, particularly preferably 0.08 mg/kg or less, and most preferably 0.06 mg/kg or less, and the minimum dose is preferably 0.005 mg/kg or more, more preferably 0.01 mg/kg or more, still more preferably 0.02 mg/kg or more, and particularly preferably 0.04 mg/kg or more.

The medicament is administered once or several times a day as required. As for administration interval, the medicament may be administered once in 2 to 14 days, preferably once in 2 to 7 clays, more preferably once in 3 to 5 days.

When the medicament of the present invention containing thrombomodulin obtained as described above was administered to acute leukemia patients who developed complications accompanied by a hypochondrial pain and weight gain after hematopoietic cell transplantation, the hypochondrial pain disappeared on the day of the administration or the next day. Further, improvement of the weight gain was observed several days after the administration of the medicament of the present invention containing thrombomodulin, and thus superior prophylactic and therapeutic effects were observed on development of complications associated with hematopoietic cell transplantation. In other words, on the basis of the effects observed in the hematological malignancy patients, it was confirmed that the medicament of the present invention was useful for prophylactic and/or therapeutic treatment of a pain associated with hematopoietic cell transplantation in a patient who underwent hematopoietic cell transplantation. When the medicament of the present invention is prepared as described above, the medicament may be prepared by a known method comprising mixing an effective amount of thrombomodulin with a pharmaceutically acceptable carrier.

The medicament of the present invention is extremely useful, because the medicament can safely achieve the superior effect without increasing the risk of hemorrhage even in a hematological malignancy patient who easily bleeds, when the patient undergoes hematopoietic cell transplantation. The aforementioned usefulness can also be supported by the fact that it has been reported that thrombomodulin, the active ingredient of the medicament of the present invention, does not exacerbate hemorrhage, but rather improves hemorrhagic conditions (Saito H. et al., J. Thromb. Haemost., 5:31-41, 2007). Examples of method for evaluating the risk of hemorrhage include measurement of clotting time such as the activated partial thromboplastin time (APTT) described by, for example, Mohri M. et al. (Mohri M. et al., Thromb. Haemost., 82:1687-93, 1999) using a specimen prepared by adding the medicament of the present invention to plasma. Examples of method for evaluating hemorrhage condition in a patient undergoing hematopoietic cell transplantation include use of amount of blood (blood transfusion) or a plasma preparation as an index.

The medicament of the present invention is useful also from a viewpoint that the medicament can be used without detriment of convenience that hematopoietic cell transplantation can be carried out with little surgical invasion in a short period of time, which is a characteristic of hematopoietic cell transplantation. Since the medicament of the present invention does not increase the risk of hemorrhage as described above, any test for determining whether the dose is appropriate is unnecessary. Examples of the test for evaluating whether a dose is appropriate include the aforementioned method for evaluating risk of hemorrhage using the clotting time as an index.

### [Explanation of Sequence Listing]

SEQ ID NO: 1: Amino acid sequence encoded by the gene used in production of TME456
SEQ ID NO: 2: Nucleotide sequence encoding the amino acid sequence of SEQ ID NO: 1
SEQ ID NO: 3: Amino acid sequence encoded by the gene used in production of TME456M
SEQ ID NO: 4: Nucleotide sequence encoding the amino acid sequence of SEQ ID NO: 3
SEQ ID NO: 5: Amino acid sequence encoded by the gene used in production of TMD12
SEQ ID NO: 6: Nucleotide sequence encoding the amino acid sequence of SEQ ID NO: 5
SEQ ID NO: 7: Amino acid sequence encoded by the gene used in production of TMD12M
SEQ ID NO: 8: Nucleotide sequence encoding the amino acid sequence of SEQ ID NO: 7
SEQ ID NO: 9: Amino acid sequence encoded by the gene used in production of TMD123
SEQ ID NO: 10: Nucleotide sequence encoding the amino acid sequence of SEQ ID NO: 9
SEQ ID NO: 11: Amino acid sequence encoded by the gene used in production of TMD123M
SEQ ID NO: 12: Nucleotide sequence encoding the amino acid sequence of SEQ ID NO: 11
SEQ ID NO: 13: Synthetic DNA for mutation used for carrying out site-directed mutagenesis

### EXAMPLES

The present invention will be explained in detail with reference to examples and test examples. However, the present invention is not limited by these examples.

The thrombomodulin of the present invention used in the test examples was prepared according to the aforementioned method of Yamamoto et al. (the method described in Japanese Patent Unexamined Publication No. 64-6219). Preparation examples thereof are described below. Safety of the thrombomodulins obtained in these preparation examples was confirmed by single and repetitive intravenous administration tests using rats and monkeys, mouse reproduction test, local irritation test, pharmacological safety test, virus inactivation test, and the like.

### Preparation Example 1

### <Obtaining Thrombomodulin>

A highly purified product was obtained by the aforementioned method. Specifically, Chinese hamster ovary (CHO) cells were transfected with a DNA encoding the amino acid sequence of SEQ ID NO: 9 (which specifically consisted of the nucleotide sequence of SEQ ID NO: 10). From the culture of the above transformant cells, a highly purified product was obtained by collecting an active fraction with a 20 mmol/L phosphate buffer (pH 7.3) containing 50 mmol/L NaCl according to the aforementioned conventional purification method. The product was further subjected to ultra filtration to obtain a thrombomodulin solution having a concentration of 11.2 mg/mL (henceforth also abbreviated as TMD123).

### <Preparation of Additive Solution>

Arginine hydrochloride (480 g, manufactured by Ajinomoto Co., Inc.) was weighed in a 10-L stainless steel vessel, and dissolved by adding 5 L of water for injection. The solution was adjusted to pH 7.3 by adding a 1 mol/L sodium hydroxide solution.

### <Preparation and Addition of Drug Solution>

The total volume of the aforementioned additive solution was put into a 20-L stainless steel vessel, 2398 mL of the TMD123 solution obtained above (corresponding to 26.88 g of soluble thrombomodulin protein, added in a 12% excess amount) was added to the above solution, and they were mixed by stirring. Water for injection was further added to the mixture up to a total volume of 12 L, and the mixture was uniformly mixed and stirred. This drug solution was subjected to filtration sterilization using a filter having a pore diameter of 0.22 μm (MCGL10S, manufactured by Millipore). The filtrate was filled in vials in a volume of 1 mL each, and the vials were half-stoppered with rubber stoppers.

### <Lyophilization>

A lyophilization process was carried out under the following conditions in the order of lyophilization→filling with nitrogen→complete stopping with a rubber stopper→screw capping to obtain a TMD123-containing preparation containing 2 mg of soluble thrombomodulin and 40 mg of arginine hydrochloride in a single vial.

### <Lyophilization Conditions>

Preliminary cooling (from room temperature to 15° C. over 15 minutes)→main cooling (from 15° C. to −45° C. over 2 hours)→retention (at −45° C. for 2 hours)→start of vacuuming (at −45° C. for 18 hours)→temperature elevation (from −45° C. to 25° C. over 20 hours)→retention (at 25° C. for 15 hours)→temperature elevation (from 25° C. to 45° C. over 1 hour)→retention (at 45° C. for 5 hours)→cooling to room temperature (from 45° C. to 25° C. over 2 hours)→filling of nitrogen for pressure recovery (up to −100 mmHg)→complete stopping→screw capping

### Preparation Example 2

Chinese hamster ovary (CHO) cells are transfected with a DNA encoding the amino acid sequence of SEQ ID NO: 11 (which specifically consists of the nucleotide sequence of SEQ ID NO: 12), a solution of thrombomodulin purified from a culture of the above transformant cells (henceforth also abbreviated as TMD123M) by the aforementioned conventional purification method is obtained, and a lyophilized preparation of TMD123M is obtained in the same manner as that described above.

### Preparation Example 3

Chinese hamster ovary (CHO) cells are transfected with a DNA encoding the amino acid sequence of SEQ ID NO: 1 (which specifically consists of the nucleotide sequence of SEQ ID NO: 2), thrombomodulin purified from a culture of the above transformant cells (henceforth also abbreviated as TME456) by the aforementioned conventional purification method is obtained, and a lyophilized preparation of TME456 is obtained in the same manner as that described above.

### Preparation Example 4

Chinese hamster ovary (CHO) cells are transfected with a DNA encoding the amino acid sequence of SEQ ID NO: 3 (which specifically consists of the nucleotide sequence of SEQ ID NO: 4), thrombomodulin purified from a culture of the above transformant cells (henceforth also abbreviated as TME456M) by the aforementioned conventional purification method is obtained, and a lyophilized preparation of TME456M is obtained in the same manner as that described above.

### Preparation Example 5

Chinese hamster ovary (CHO) cells are transfected with a DNA encoding the amino acid sequence of SEQ ID NO: 5 (which specifically consists of the nucleotide sequence of SEQ ID NO: 6), thrombomodulin purified from a culture of the above transformant cells (henceforth also abbreviated as TMD12) by the aforementioned conventional purification method is obtained, and a lyophilized preparation of TMD12 is obtained in the same manner as that described above.

### Preparation Example 6

Chinese hamster ovary (CHO) cells are transfected with a DNA encoding the amino acid sequence of SEQ ID NO: 7 (which specifically consists of the nucleotide sequence of SEQ ID NO: 8), thrombomodulin purified from a culture of the above transformant cells (henceforth also abbreviated as TMD12M) by the aforementioned conventional purification method is obtained, and a lyophilized preparation of TMD12M is obtained in the same manner as that described above.

### Test Example 1

### <Effect in Philadelphia Chromosome-Positive Acute Lymphatic Leukemia (Ph-Positive ALL) Patient Received Hematopoietic Cell Transplantation>

To a 44-year old female developed Ph-positive ALL, a chemotherapy including use of imatinib and a conditioning regimen with fludarabine and melphalan were given, and then hematopoietic cell transplantation was performed from a one-locus HLA-mismatched elder brother. Tacrolimus and mycophenolic acid were used for a prophylactic treatment for GVHD. The patient was diagnosed to be developing SOS because weight gain and right hypochondrial pain were observed on the 8th day after the transplantation. Administration of an ATIII preparation (1,500 U/day) and defibrotide was started because increase of FDP and PT was observed. However, the weight gain, right hypochondrial pain, and hypercoagulation state were not improved, and under the above conditions, TMD123 administration was started from the 12th day after the transplantation. Before the start of the TMD123 administration, the bilirubin value and GPT value were found to be normal. The right hypochondrial pain disappeared after 3 to 4 hours from the start of the TMD123 administration. The increased weight began to be decreasing on the 3rd day of the TMD123 administration, and decreased down to a weight comparable to the weight before the hematopoietic cell transplantation two weeks after the end of the TMD123 administration. FDP was decreased to a normal level on the next day of the end of the TMD123 administration. PT was decreased to a normal level 3 days after the end of the TMD123 administration. Even after the end of TMD123 administration, the bilirubin value and GPT value were found to be normal.

As TMD123, a product prepared in the same manner as that of Preparation Example 1 was used.

### Test Example 2

### <Effect in Adult T-Cell Leukemia (ATL) Patient Received Hematopoietic Cell Transplantation>

To a 48-year old female developed ATL, a chemotherapy and a conditioning regimen with a large amount of cyclophosphamide and TBI were given, and then hematopoietic cell transplantation was performed from a completely HLA-matched younger sister. Cyclosporin and methotrexate were administered as a prophylactic treatment for GVHD. Increases of FDP and PT were observed from the 7th day after the transplantation, and an ATIII preparation was administered from the 9th day after the transplantation. The patient was diagnosed to be developing SOS because weight gain and hepatomegaly accompanied by oppressive pain were observed on the 18th day after the transplantation, and then administration of TMD123 was started. Before the end of the TMD123 administration, the bilirubin value and GPT value were found to be normal. The right hypochondrial pain disappeared on the next day of the TMD123 administration. The weight gain was stopped by the TMD123 administration, and the body weight began to be decreasing on the 6th day of the TMD123 administration, and decreased down to a weight comparable to the weight before the hematopoietic cell transplantation at the end of the TMD123 administration. FDP was decreased to a normal level 2 days after the end of the TMD123 administration. PT was decreased to a normal level on the day of the end of the TMD123 administration. Even after the end of the TMD123 administration, the bilirubin value and GPT value were found to be normal.

As TMD123, a product prepared in the same manner as that of Preparation Example 1 was used.

1. A method for prophylactic and/or therapeutic treatment of a pain associated with hematopoietic cell transplantation, which comprises the step of administrating thrombomodulin to a mammal.

2. The method according to claim 1, wherein the thrombomodulin is a soluble thrombomodulin.

3. The method according to claim 1 or 2, wherein the thrombomodulin is a peptide obtainable from a transformed cell prepared by transfecting a host cell with a DNA encoding the amino acid sequence of SEQ ID NO: 9 or SEQ ID NO: 11.

4. The method according to claim 1, wherein the thrombomodulin is a peptide having the sequence from the position 19 to 516 in either of SEQ ID NO: 9 or SEQ ID NO: 11, or a peptide having an amino acid sequence comprising substitution, deletion, or addition of one or more amino acid residues of the amino acid sequence of the aforementioned peptide and having thrombomodulin activity.

5. The method according to claim 1, wherein the hematopoietic cell transplantation is hematopoietic cell transplantation for hematological malignancy.

6. The method according to claim 1, wherein the pain associated with hematopoietic cell transplantation is a pain developed with any one disease selected from the group consisting of graft versus host disease, sinusoidal obstruction syndrome, and transplantation-associated thrombotic microangiopathy.

7. The method according to claim 1, wherein the pain associated with hematopoietic cell transplantation is developed in any one region selected from the group consisting of the eyeball, oral cavity, abdomen, and hypochondrium.

8. The method according to claim 1, which comprises the step of administering thrombomodulin together with at least one therapeutic agent selected from the group consisting of an immunosuppressant and a therapeutic agent for hematological malignancy.

9. A method for improving hematopoietic ability at the time of hematopoietic cell transplantation for hematological malignancy, which comprises the step of administrating thrombomodulin to a mammal.
